(19) United States
(12) Patent Application Publication
(10) Pub. No.: US 2019/0295702 A1
(43) Pub. Date: Sep. 26, 2019

(54) PATIENT FILTERING BASED ON LIKELIHOOD OF SIDE EFFECTS

(71) Applicants: Ritankar Das, Hayward, CA (US); Samson Mataraso, Hayward, CA (US); Jacob Calvert, Hayward, CA (US)

(72) Inventors: Ritankar Das, Hayward, CA (US); Samson Mataraso, Hayward, CA (US); Jacob Calvert, Hayward, CA (US)

(21) Appl. No.: 15/934,986

(22) Filed: Mar. 24, 2018

(51) Int. Cl. G16H 20/10; G16H 10/60

(52) U.S. Cl. CPC G16H 20/10; G16H 10/60

(57) ABSTRACT

Methods for identifying to which patients a drug should be administered, based on underlying drug mechanism of action, are provided. Multiple types of data, including demographic, physiological, treatment, and clinical notes data, can be used to train a classification component. Multiple patient populations can be used as sources of patient data for training classification component. Data input requirements, dimensionality, and performance metrics may be optimized.

### FIELD OF THE INVENTION

The present disclosure generally relates to patient monitoring and diagnosis, and in particular to the design and implementation of clinical trials and the administration of drugs.

### BACKGROUND OF THE INVENTION

Clinicians have traditionally made decisions regarding which medication to prescribe for a patient's health condition or illness based on medication information that they have memorized or can quickly look up in a reference guide. The clinician may have learned about a given kind of medication, for example, a specific anti-inflammatory agent, from a medical journal, advertisement, educational lecture, or other means, Generally, drugs target specific physiologic pathways through their mechanism of action, and a disease which presents very similarly may be caused by different physiologic pathways. In certain heterogeneous disease populations (for example, sepsis, although there are many others), it would be valuable to know which drug to pick based on the underlying physiologic insult causing the disease. Even outside of heterogeneous disease populations, patients with identical manifestations of a condition likely have differences in the rest of their physiology. As such, drugs are likely to affect patients differently. Each drug has a certain mechanism of action that drives its ability to treat disease. These mechanisms of action can interact in detrimental ways to different physiological phenomena in a human body. Currently, there are limited ways to detect these phenomena in advance to avoid giving these drugs to such patients, preventing potentially fatal side effects.

### SUMMARY OF THE INVENTION

The presently disclosed embodiments are directed to solving one or more of the problems presented in the prior art, described above, as well as providing additional features that will become readily apparent by reference to the following detailed description when taken in conjunction with the accompanying drawings.

In an embodiment, the disclosure provides a method of filtering out a patient likely to suffer from a side effect from drug treatment, which includes acquiring patient data from a plurality of patients; comparing the acquired patient data to classified anonymized patient health record data; and filtering out the patient from the plurality of patients, who is likely to suffer from a side effect from drug treatment based on whether or not the drug's mechanism of action is likely to cause a side effect in treating a specific manifestation of the patient's condition as evidenced by the classified anonymized patient health record data.

In another embodiment, the disclosure provides a method of filtering out a patient likely to suffer from a side effect from drug treatment, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

In another embodiment, the disclosure provides a method of filtering out a patient likely to suffer from a side effect from drug treatment, wherein the anonymized patient health record data reflects the nature of the patient population served by the hospital or clinic in terms of patient demographics, rates of disease incidence, and/or treatment practices.

In another embodiment, the disclosure provides a method of filtering out a patient likely to suffer from a side effect from drug treatment, wherein the anonymized patient health record data is sourced from a database of the plurality of patients, a database of one or more care centers and patient populations, or from a database of multiple care centers and patient populations.

In another embodiment, the disclosure provides a method of filtering out a patient likely to suffer from a side effect from drug treatment, wherein the anonymized patient health record data is collected at a standard interval.

In another embodiment, the disclosure provides a method of filtering out a patient likely to suffer from a side effect from drug treatment, wherein the anonymized patient health record data includes at least patient labeled positively with respect to the designated gold standard which identifies that patient as having a physiologic insult which will interact negatively with the mechanism of action of a specific drug.

In another embodiment, the disclosure provides a method of filtering out a patient likely to suffer from a side effect from drug treatment, wherein the data contain at least one patient labeled positively with respect to a designated gold standard which specifies the patient as progressing through a specific disease pathway.

In another embodiment, the disclosure provides a method of filtering out a patient likely to suffer from a side effect from drug treatment, wherein the anonymized patient health record data continually improve as new data becomes available.

In another embodiment, the disclosure provides a method of filtering out a patient likely to suffer from a side effect from drug treatment, wherein the classified anonymized patient health record data includes an operating point that balances measurements of specificity and sensitivity in order to effectively treat as many patients as possible.

In another embodiment, the disclosure provides a method of filtering out a patient likely to suffer from a side effect from drug treatment, wherein the drug treatment is administration of drotrecogin alfa, tozadenant, vosaroxin, momelotinib or resatorvid or any salt, acid, base, hydrate, solvate, ester, isomer, polymorph, metabolite or prodrug thereof.

In another embodiment, the disclosure provides a method of using a machine learning algorithm for filtering out a patient likely to suffer from a side effect from drug treatment, which includes acquiring anonymized patient health record data from a plurality of patients; comparing acquired patient data to the acquired anonymized patient health record data; and filtering out the patient from the plurality of patients, who is likely to suffer from a side effect from drug treatment based on whether or not the drug's mechanism of action is likely to cause a side effect in treating a specific manifestation of the patient's condition as evidenced by the classified anonymized patient health record data.

In another embodiment, the disclosure provides a method of using a machine learning algorithm for filtering out a patient likely to suffer from a side effect from drug treatment, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

In another embodiment, the disclosure provides a method of using a machine learning algorithm for filtering out a patient likely to suffer from a side effect from drug treatment, wherein the anonymized patient health record data reflects the nature of the patient population served by the hospital or clinic in terms of patient demographics, rates of disease incidence, and/or treatment practices.

In another embodiment, the disclosure provides a method of using a machine learning algorithm for filtering out a patient likely to suffer from a side effect from drug treatment, wherein the anonymized patient health record data is sourced from a database of the plurality of patients, a database of one or more care centers and patient populations, or from a database of multiple care centers and patient populations.

In another embodiment, the disclosure provides a method of using a machine learning algorithm for filtering out a patient likely to suffer from a side effect from drug treatment, wherein the anonymized patient health record data is collected at a standard interval.

In another embodiment, the disclosure provides a method of using a machine learning algorithm for filtering out a patient likely to suffer from a side effect from drug treatment, wherein the labeled patient data includes a positive label for a gold standard which represents a specific progression through the disease pathway.

In another embodiment, the disclosure provides a method of using a machine learning algorithm for filtering out a patient likely to suffer from a side effect from drug treatment, wherein the at least one gold standard patient data includes a specific progression through the disease pathway.

In another embodiment, the disclosure provides a method of using a machine learning algorithm for filtering out a patient likely to suffer from a side effect from drug treatment, wherein the anonymized patient health record data continually improve as new data becomes available.

In another embodiment, the disclosure provides a method of using a machine learning algorithm for filtering out a patient likely to suffer from a side effect from drug treatment, wherein the classified anonymized patient health record data includes an operating point that balances measurements of specificity and sensitivity in order to effectively treat as many patients as possible.

In another embodiment, the disclosure provides a method of using a machine learning algorithm for filtering out a patient likely to suffer from a side effect from drug treatment, wherein the drug treatment is administration of drotrecogin alfa, tozadenant, vosaroxin, momelotinib or resatorvid or any salt, acid, base, hydrate, solvate, ester, isomer, polymorph, metabolite or prodrug thereof.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, size, or applicability of the disclosure. It should be noted that for clarity and ease of illustration these drawings are not necessarily made to scale.

FIG. 1 illustrates an embodiment of the different types of data that can be used that are relevant to the classification or prediction task;

FIG. 2 illustrates an embodiment of the possibilities for training on different populations of data;

FIG. 3 illustrates an embodiment of a gold standard determination of positive-class and negative-class labels;

FIG. 4 illustrates an embodiment of decision-making processes; and

FIG. 5 illustrates an embodiment of an inclusion chart of a clinical trial run with an algorithmic companion diagnostic.

### DETAILED DESCRIPTION OF EXEMPLARY EMBODIMENTS

The following description is presented to enable a person of ordinary skill in the art to make and use embodiments described herein. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the disclosure. The word “exemplary” is used herein to mean “serving as an example illustration.” Any aspect or design described herein as “exemplary” is not necessarily to be construed as preferred or advantageous over other aspects or designs. Thus, the present disclosure is not intended to be limited to the examples described herein and shown but is to be accorded the scope consistent with the claims.

As used herein, reference to any biological drug includes any fragment, modification or variant of the biologic, including any pegylated form, glycosylated form, lipidated form, cyclized form or conjugated form of the biologic or such fragment, modification or variant or prodrug of any of the foregoing. As used herein, reference to any small molecule drug includes any salt, acid, base, hydrate, solvate, ester, isomer, or polymorph thereof or metabolite or prodrug of any of the foregoing.

It should be understood that the specific order or hierarchy of steps in the process disclosed herein is an example of exemplary approaches. Based upon design preferences, it is understood that the specific order or hierarchy of steps in the processes may be rearranged while remaining within the scope of the present disclosure. Any accompanying method claims present elements of the various steps in a sample order and are not meant to be limited to the specific order or hierarchy presented.

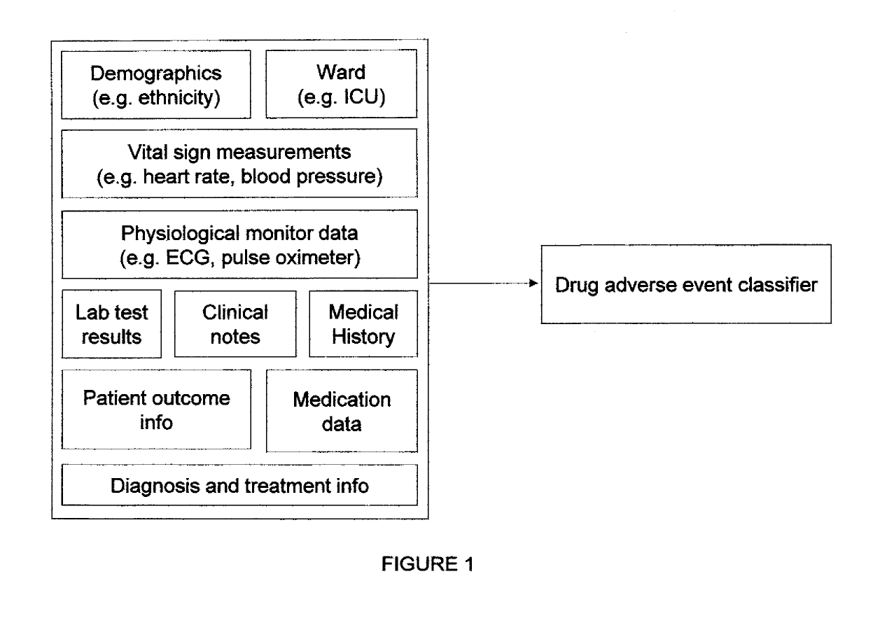

FIG. 1 illustrates an embodiment of the different types of data that can be used that are relevant to the classification or prediction task. The use of machine learning techniques necessitates the availability of data relevant to the classification or prediction task, on which to train the machine learning algorithm. In the context of the current disclosure, these data are typically anonymized patient health records, which can include amongst other information (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and (x) patient medical history. Any or all of these types of data, along with other types of patient health information, can serve as inputs to the training procedure associated with a machine learning algorithm.

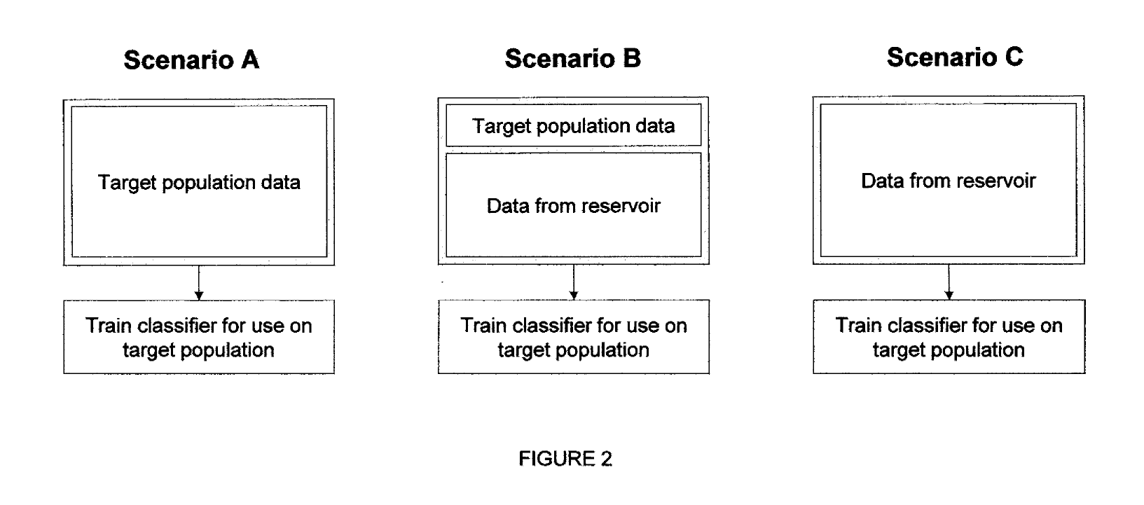

FIG. 2 illustrates an embodiment of the possibilities for training on different populations of data. Patient health record information can be collected and stored by the hospitals or clinics which deliver patient care. In this case, the health record data may reflect the nature of the patient population served by the hospital or clinic, in terms of patient demographics, rates of disease incidence, treatment practices, and other information potentially relevant to a prediction or classification task. Accordingly, when using a machine learning algorithm to develop a prediction or classification tool for use at a hospital or clinic, it may be useful to train with data from the relevant patient population or data from a similar population.

Patient health record data for training a machine learning algorithm can be sourced from a pre-existing or reservoir of data, consisting of anonymized health records from one or more care centers and patient populations, typically with some variability in the types and amount of data that are available for patients in the data set. Alternatively, health record data can be obtained from multiple care centers and patient populations. For example, the Medical Information Mart for Intensive Care III (MIMIC-III) is a publicly-accessible database of anonymized patient health record information, collected from Beth Israel Deaconess Medical Center (Boston, Mass.) between 2001 and 2012, which contains many of the aforementioned types of health data for tens of thousands of patients. A database like MIMIC-III would contrast with patient health record data available from the Veterans Health Administration, for example, which consists of many more health centers, spread across many states and cities. Accordingly, the types and resolution of health data available from such a data set would likely vary more.

When training a machine learning algorithm, it is typically ideal to train on a data set collected from the same population on which the resulting tool is intended to be applied. If there are sufficient training data from the target care center or population, the training procedure can proceed without modification as specified by the machine learning algorithm. If, however, there are not sufficient available data, the training procedure may be modified to rely on both a reservoir of health record data, as well as a small collection of clinic- or population-specific data; alternatively, the training procedure may rely entirely on a reservoir of data. A typical way to modify the training procedure in the former case is with the techniques of transfer learning, wherein the machine learning algorithm is first trained on a reservoir of data, before being trained further on the target dataset in such a way as to emphasize the examples it contains.

Ideally, all measurements relevant to a prediction or classification task are measured frequently, at a standard interval (e.g. one measurement every hour). However, patient health record data include types of data with varying frequencies of measurement and, as such, it is often convenient to standardize the frequencies with which new measurements are assessed by the prediction or classification tool resulting from the training procedure. For example, to produce a new patient classification every hour, the time series of measurements may be partitioned or “binned” into one-hour increments and relayed to the classifier accordingly.

As there will likely be bins during which no new measurement is available for a particular patient and type of data, it is standard to implement a data imputation scheme, whereby available data are used to fill-in missing data. The simplest such imputation method is a “carry-forward”rule, where the most recent measurement for a particular input (e.g. a heart rate measurement) is used in subsequent empty bins. There are other, more complicated methods for data imputation, including the filling of empty bins with the running average of the measurements of the relevant input, or inferring the missing value from a patient with a quantitatively similar trajectory of measurements.

It is also the case that sometimes multiple measurements of the same clinical variable are available within the same binning period. In this case, the frequency of measurements can be standardized by replacing the multiple measurements with the average of their values.

Supervised machine learning algorithms require labeled training data to identify the patterns in the data from which labels can be inferred. For example, to train a classifier for a sepsis prediction from patient health record data, each patient must be assigned a positive or negative label, respectively indicating whether the patient did or did not have sepsis. Typically, before using unlabeled patient data with a supervised learning algorithm, the relevant label is assigned to the patient unambiguously in terms of the data that are available for that patient. This unambiguous way of assigning a label is called a gold standard.

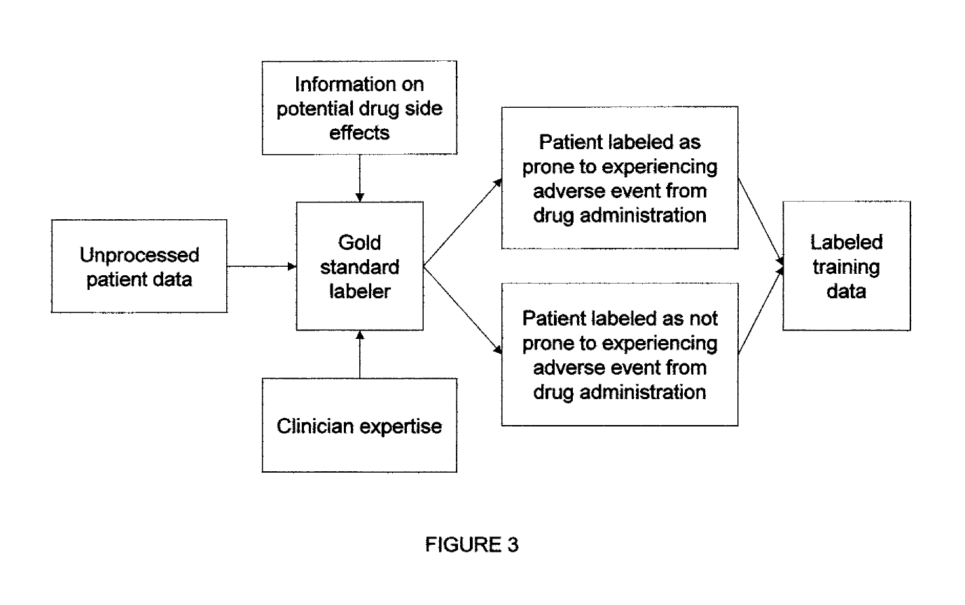

FIG. 3 illustrates an embodiment of a gold standard determination of positive-class and negative-class labels. In many cases, there is no universally agreed-upon gold standard for a label of medical relevance. Accordingly, a choice of gold standard may rely on a combination of standards of medical practice, correlative analyses, data availability constraints, and clinician expertise.

In the context of developing a tool for identifying patients for whom a drug may have deleterious effects, a gold standard would produce a label indicating if a given patient is likely to suffer side effects from a certain drug. While it may seem like only patients with conditions in the drug's indication should be included, misdiagnosis as well as off-label use could be extremely harmful if this restriction is used in the development of a gold standard. As such, when labeling data, it is important to use all patient data available. One example is the drug drotrecogin alfa (activated) (DrottA, Xigris, Activated human protein C, Eli Lilly and Company), a drug developed for the treatment of sepsis. One of the problems with drotrecogin alfa (which ultimately led to an unfavorable risk-benefit profile) was it caused intracranial hemorrhaging (ICH). Some patients, such as those with coagulopathy, may have their risk of ICH exacerbated. Consequently, a gold standard that is an approximate proxy for coagulopathy could lead to development of an algorithm that is able to identify patients likely to suffer from ICH, and allow clinicians to avoid giving them the drug, improving the risk-benefit profile. Thus, in an embodiment, the disclosed algorithms can determine which patients have coagulopathy, allowing physicians to avoid prescribing drotrecogin alfa which has a higher chance of resulting in intracranial hemorrhaging in these patients.

Many antibiotics have nephrotoxic side effects, and although they may be effective at killing bacteria, these side effects are too harmful and result in an unfavorable risk-benefit profile. The disclosed algorithms could select patients that are likely to suffer from these nephrotoxic side effects, thus preserving the efficacious ability of the drug in the subset of patients who will not be excessively harmed by the drug. 2. Thus, the disclosed algorithms can determine which patients are at high risk for nephrotoxic side effects in patients, which will allow physicians to avoid giving these patients antibiotics.

Many chemotherapy drugs have immunosuppressive side effects. As such, a dysregulated response to infection is of great concern. By detecting signs of infection and immuno-dysregulation early, the disclosed algorithmic approach can enable physicians to make informed decisions regarding the pros and cons of pausing chemotherapy to allow a patient to fight infection. Thus, the disclosed algorithms can detect early signs of imrnunodysregulation, which is an adverse event commonly associated with chemotherapy. Physicians can closely monitor these patients for potential signs of sepsis. Vosaroxin ((AG-7352, SPC-595, SNS 595, voreloxin, 7-[(3S,4S)-3-methoxy-4-(methylamino)-1-pyrrolidinyl]-4-oxo-1-(1,3-thiazol-2-yl)-1,4-dihydro-1,8-naphth-yridine-3-carboxylic acid) is an example drug. The disclosed algorithms can also detect early signs of sepsis, which is an adverse event associated with many drugs. Clinicians can more carefully prescribe these drugs (e.g. tozadenant (SYN115, 4-hydroxy-N-[4-methoxy-7-(4-morpholinyl)-2-benzothiazolyl]-4-methyl-1-piperidinecarboxamide, Biotie Therapies) to patients at high risk for sepsis.

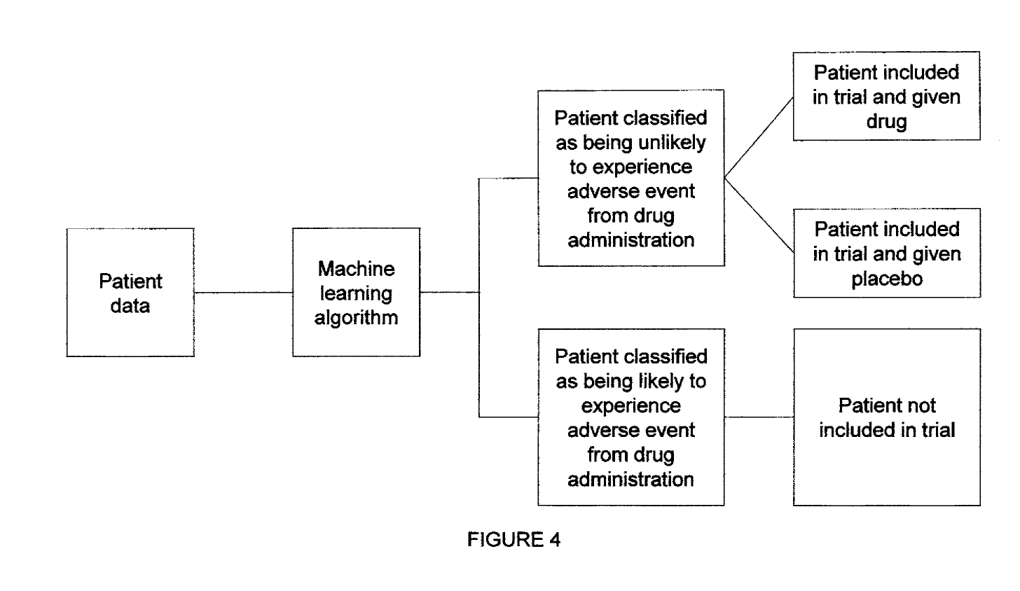

FIG. 4 illustrates an embodiment of decision-making process, which includes (1) machine-learning-based tool determines that patient is in subset of disease progression pathways that would not interact negatively with mechanism of action target, (ii) patient is allocated uniformly-at-random to experimental or control groups of trial relevant clinicians are alerted to positive-class classification of patient, (iii) relevant clinicians administer drug to patient on the basis of positive-class classification.

The machine learning procedure identifies which features of the data set are most important for the classification or prediction task under consideration. Typically, in the context of the current disclosure, the features are the clinical variables (e.g. vital signs, lab test results), as well as their correlations (e.g. correlation between heart rate and blood pressure) and trends over time (e.g. differences in measurements taken at the beginning and end of a time window). However, it may also be the case that the features consist of all the data points of a patient's stay or, contrastingly, exclusively derivatives thereof.

If too many features are used in the learning procedure, training can be slow and may overfit the data. Overfitting leads to the appearance of good prediction performance, when tested with the data set on which it was trained, but results in poor generalization to other data sets (i.e. other patient populations). One way to prevent overfitting is by reducing the dimensionality, or number of features, included in the training procedure. Preliminary training and testing can identify those features which are most important to the prediction process; less important features can then be removed from the training procedure.

The result of the training procedure is, in one form or another, a weighting of the features which can then be used to make predictions on new examples, subject to the features first being constructed from the new data. For classifying aspects of patient health, weighting the various features often leads to a numerical score which reflects the extent to which a given patient is believed to belong to a particular class. By placing a threshold on the score (e.g. patients whose scores are above 10 are determined to have sepsis; those with scores below 10 do not).

These machine learning algorithms can not only utilize information about a patient's current medical state, but also contextualize measurement information. The algorithm contextualizes by looking at deviation from a prior normal. Although there are medically accepted reference ranges for normal values of certain measurements, by analyzing prior measurements the algorithms determine what is normal for a specific patient. Consider, for example, a drug with a side effect that causes hypertension. Although a systolic blood pressure of 120 mmHg is often considered normal, it represents hypertension in a patient who is regularly hypotensive. As such, the algorithm can be trained to avoid drugs with potentially hypertensive side effects, as it relates to the context of the patient's general health.

Training a machine learning algorithm must be done cognizantly, as the performance of the algorithm depends heavily on (i) the data used to train the algorithm and (ii) the technique chosen. The data used to train the algorithm must labeled with a gold standard as described in the prior sections. Further, previously described various imputation and feature selection methods must be used to ensure the data is as well formed as possible. Well-formed data allow training processes to identify the best features for predicting patient response to various drugs.

Multiple learning techniques were tested when developing the companion algorithms and utilize the one that produces the best area under the receiver operating characteristic curve. Simple techniques can be tested such as linear regression, which attempts to find the best equation for a linear regression to fit to the data. More complicated techniques can be tested, such as gradient boosted trees. Gradient boosted trees utilize multiple weak prediction models, in this case, decision trees. Decision trees are rule-based models which assign what is in effect a score based on an established set of rules. When combining many decision trees through gradient boosting, very robust predictions are often seen.

As the disclosed algorithms make suggestions about which patient should be receiving treatment based on whether or not a drug's mechanism of action would be more efficacious than deleterious in treating the specific manifestation of their condition and as patients are treated accordingly, the patient's health typically improves; however, it may not. In order to reduce false positives (which could cause alarm fatigue), there are inevitably patients that are not treated which should be. Likewise, in order to treat as many patients as possible, the disclosed algorithm sometimes suggests treating patients for whom the drug is harmful. This balance is achieved by selecting an appropriate operating point, which balances these two measurements (specificity and sensitivity).

As the disclosed algorithms run and patients are treated, more data is generated. Another training technique utilized is called online learning. Online learning allows algorithms to continually improve themselves as new data become available. In this context, the disclosed algorithm can be able to learn from its own mistakes. If it suggests prescribing a drug to a patient that ultimately has an underlying physiologic insult which responds negatively to the drug's mechanism of action, that patient can become part of the training data and improve the algorithm's future predictions.

Selection of an Operating Point (could be included in the training section). There are many settings (indicated as points along an ROC curve); the selection of an operating point is at the discretion of the user. It is a trade-off of sensitivity and specificity; this connects with the number of alerts a user would like to have over a period of time.

Although these algorithms are developed to predict a binary outcome—whether or not a patient will respond positively to a drug or if they will suffer from adverse events—they are trained to output some score along a scale. The binary outcome is achieved by choosing an operating point. However, by selecting multiple operating points, the disclosed algorithms can be generalized to scenarios in which there are multiple options. Depending on the range that a patient's score falls within, a specific course of clinical treatment can be suggested. Consider for example drug dosing.

If a patient is likely to have a certain physiological phenomenon that would interact negatively with a drug's mechanism of action, but the drug also has a strong potential to treat a patient's condition, the disclosed algorithms can be designed to administer a partial dose of the drug, monitor the patient, and determine if they improve. Whether or not the patient improves allows the algorithm to better determine whether or not the patient had the specific physiologic phenomenon and suggest appropriate treatment. Further, this label can be used in conjunction with the online learning scheme described above. However, if the disclosed algorithm's score for a particular patient falls outside of this grey area (i.e. far from the operating point that would be established in a binary decision), administering a stronger dose of the drug can be chosen.

Dosing differences are very common in Phase III clinical trials—many trials consist of three trial arms: a placebo, a low dose, and a high dose. Often times adverse events are more common in the higher dose arm. While placebo vs active arm is typically randomized, the disclosed algorithms can be used to assign in which dosing group patients that have been assigned to the active arm should be placed.

In clinical settings, the disclosed algorithms can be implemented directly within an EHR. This direct implementation allows for algorithms to process data in real time from patients as it is entered into their medical records. Further, alerts can be displayed directly to clinicians in a patient's chart. However, external alerts, such as phone calls, are also possible through integration with automated calling APIs. When the disclosed algorithms detect that a patient has a certain condition that has manifested with a physiology that is conducive to being treated with a drug, it can alert a clinician that (i) they have a certain condition and (ii) a certain drug should be prescribed. However, it can include a warning if the patient is likely to suffer from an adverse event so a clinician can make an informed decision about the best course of treatment.

One example may be the Parkinson's drug tozadenant. A phase III trial found patients who received tozadenant developed sepsis at higher rates than those who received a placebo treatment. By monitoring a patient, it is possible for the disclosed algorithm to determine likelihood of developing sepsis. This probability of developing this adverse event can be presented to a clinician and weighed against the potential benefit of the drug, allowing a clinician to make a more informed decision about whether or not the drug is to be administered.

Another example is the acute myeloid leukemia (AML) drug vosaroxin (Sumitomo Dainippon Pharma Co., Ltd.). A phase III trial found that some patients receiving vosaroxin died within 30 days of beginning treatment due to infection or sepsis. The disclosed invention could be trained to detect patients likely to develop sepsis. If a patient is likely to develop sepsis, vosaroxin would not be administered to them. If the patent has already begun treatment, a clinician can monitor the patient more closely for early warning signs of sepsis and can wean the patient off vosaroxin if necessary.

Additionally, consider the Janus kinase inhibitor class of drugs, such as momelotinib (N-(cyanomethyl)-4-{2-[4-(morpholin-4-yl)anilino]pyrimidin-4-yl}benzarnide, Gilead Sciences, Inc.). Often times Janus kinase inhibitors cause adverse events such as anemia, neutropenia, and thrombocytopenia. By training the disclosed algorithm to detect patients likely to develop these adverse events, it is possible to avoid administering Janus kinase inhibitors to these patients, thus reducing the rate of adverse events and improving patient outcomes. Often times Janus kinase inhibitors cause adverse events such as anemia, neutropenia, and thrombocytopenia. By training the disclosed algorithm to detect patients likely to develop these adverse events, it is possible to avoid administering Janus kinase inhibitors to these patients, thus reducing the rate of adverse events and improving patient outcomes.

The disclosed algorithms can be used to improve clinical trials. Often times, drugs are effective, but their results in clinical trials are underpowered. By selecting patient responders that align with the mechanism of action of a drug, this efficacy signal can be strengthened, resulting in a successful trial. In a clinical trial, the disclosed algorithms can be used as inclusion criteria. Currently, patients are enrolled in a trial if they meet a rule based criteria, which often attempts to, in a rudimentary fashion, detect the physiological mechanism the drug treats. However, an algorithmic inclusion-criteria provides a more robust way of confirming that a certain patient's condition can have a favorable risk-benefit profile with respect to their likelihood of suffering some side effect. Upon a positive result from the disclosed algorithms, the algorithms cart alert a patient's clinicians that the patient (i) has a condition which makes them eligible for enrollment in a clinical trial and (ii) their health is likely to benefit by the drug being tested in their trial.

Increasingly, clinical trials utilize adaptive trial designs. Adaptive trial designs begin by looking at a drug's effect in a smaller subgroup of patients to determine likely effect size. Then, as needed, the trial is expanded to more patients to show statistical significance. A smaller initial trial size allows trial coordinators to terminate the trial if initial results show either futility or efficacy. The algorithms described can be used to improve adaptive trial design. By constantly performing interim analyses it can continuously update needed trial size to show efficacy, preventing unnecessary costs due to over enrollment in the trial. Further, the ability to select responders results in a stronger efficacy signal, meaning less patients need be enrolled to show statistical significance. As such, trial size and length can decrease, resulting in substantial cost reduction.

Fundamentally, these algorithms are a method of balancing patient response with potential harm. They serve as companion diagnostics in the same way that a more traditional biomarker or genetic test would. However, they improve upon these methods in multiple ways. Companion diagnostics such as traditional biomarkers and genetic tests require an active order from a clinician, which can not only take time but also may be overlooked. An algorithmic companion diagnostic is passive, and always monitoring patients, which means as soon as they develop some physiologic condition that can be treated by a drug, they are diagnosed thusly. Most notably, there are rarely companion diagnostics for detecting adverse events. When a patient meets some criteria for drug administration, they are generally given a drug unless there are obvious warning signs that they would suffer from adverse events. A passive algorithm continuously assessing risk of adverse events as they relate to the potential benefit of a drug ensures that patients that only truly benefit from a drug receive it.

Further, machine learning is a powerful tool for prediction. By analyzing patient trajectories, the disclosed algorithms have the ability to predict the onset of disease, in addition to detecting it. Therefore, the companion diagnostics can be able to diagnose conditions earlier.

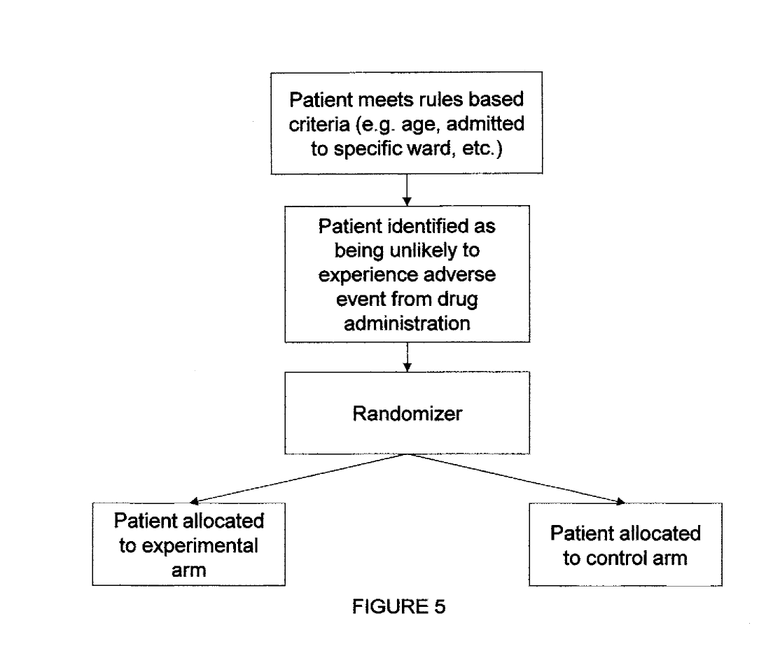

FIG. 5 illustrates an embodiment of an inclusion chart of a clinical trial run with an algorithmic companion diagnostic. Algorithms can also be developed with data. These data are already collected at thousands of hospitals across the country. There are no expensive R&D procedures necessary as are often the case with biomarkers and genetic tests. Further, they can be validated on partitioned data—no in vitro tests are required. Lastly, the ability to analyze features which represent more complicated bodily functions (e.g. by being composed of multiple vital signs and lab tests), leads to these algorithms typically having more discriminatory power. Because a positive risk-benefit profile from the algorithm is an inclusion criterion for the trial, it would become part of the drug's indication upon regulatory approval.

Because the specific physiologic phenomena (and thus the gold standards used for labeling data) are often not explicit outcomes present in the EHR, there is a substantial barrier to acquiring labeled data that can be used to train these algorithms. This issue can be solved in a number of ways. First, clinicians can hand-label charts for the presence of conditions which may have negative interactions with the mechanism of action of the drug. Manual labeling of charts can be incredibly valuable for creating a training dataset which is accurately labeled. Using these data as a base can allow an algorithm to be trained enough to produce good results, which can be further improved by online learning.

Additionally, drugs often fall into a specific class, for example resatorvid (TAIL-242, ethyl (6R)-6-[(2-chloro-4-fluorophenyl)sulfamoyl]cyclohexene-1-carboxylate), Takeda Pharma-ceutical Company, Ltd.) and eritoran (([(2R,3R,4R,5S,6R)-4-Decoxy-5-hydroxy-6-[[(2R,3R,4R,S5,6R)-4-[(3R)-3-methoxydecoxy]-6-(methoxymethyl)-3-[[(Z)-octadec-11-enoyl]-amino]-5-phosphonatooxyoxan-2-yl]oxymethyl]-3-(3-oxotetradecanoylamino)oxan-2-yl] phosphoric acid), Eisai Inc.) are both TLR-4 inhibitors, Because drugs within the same class typically have identical or similar mechanisms of action, transfer learning techniques can be applied utilizing data from patients who have received drugs of the same class.

After implementing a classifier resulting from the training procedure, it may be desirable to update the classifier to reflect different priorities of use or to reflect new patient data that have become available for training. Retraining can be completed in batches, that is, by performing the training procedure on an updated training set and choosing an operating point to reflect the use priorities (i.e. picking the sensitivity and specificity of alerts clinicians can expect to receive, which determines the number of alerts clinicians can expect to receive) in the same way as was originally done. Retraining can also be completed continuously as new data become available using an online machine learning technique.

Algorithmic stratification such as the method described in this patent may result in only a small group of patients being eligible to receive a drug, as disease populations (especially heterogeneous ones) may be partitioned into more specific manifestations of a disease. However, the FDA provides certain benefits (i.e. Orphan Drug Designation) to such conditions that affect only a small portion of the population.

While the inventive features have been particularly shown and described with reference to preferred embodiments thereof, it will be understood by those in the art that the foregoing and other changes may be made therein without departing from the sprit and the scope of the disclosure. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

What is claimed is:

1. A method of filtering out a patient likely to suffer from a side effect from drug treatment, comprising:
acquiring patient data from a plurality of patients;
comparing the acquired patient data to classified anonymized patient health record data; and
filtering out the patient from the plurality of patients, who is likely to suffer from a side effect from drug treatment based on whether or not the drug's mechanism of action is likely to cause a side effect in treating a specific manifestation of the patient's condition as evidenced by the classified anonymized patient health record data.

2. The method of claim 1, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

3. The method of claim 1, wherein the anonymized patient health record data reflects the nature of the patient population served by the hospital or clinic in terms of patient demographics, rates of disease incidence, and/or treatment practices.

4. The method of claim 1, wherein the anonymized patient health record data is sourced from a database of the plurality of patients, a database of one or more care centers and patient populations, or from a database of multiple care centers and patient populations.

5. The method of claim 1, wherein the anonymized patient health record data is collected at a standard interval.

6. The method of claim 1, wherein the anonymized patient health record data includes at least patient labeled positively with respect to the designated gold standard which identifies that patient as having a physiologic insult which will interact negatively with the mechanism of action of a specific drug.

7. The method of claim 6, wherein the data contain at least one patient labeled positively with respect to a designated gold standard which specifies the patient as progressing through a specific disease pathway.

8. The method of claim 1, wherein the anonymized patient health record data continually improve as new data becomes available.

9. The method of claim 1, wherein the classified anonymized patient health record data includes an operating point that balances measurements of specificity and sensitivity in order to effectively treat as many patients as possible.

10. The method of claim 1, wherein the drug treatment is administration of drotrecogin alfa, tozadenant, vosaroxin, momelotinib or resatorvid.

11. A method of using a machine learning algorithm for filtering out a patient likely to suffer from a side effect from drug treatment, comprising:
acquiring anonymized patient health record data from a plurality of patients;
comparing acquired patient data to the acquired anonymized patient health record data; and
filtering out the patient from the plurality of patients, who is likely to suffer from a side effect from drug treatment based on whether or not the drug's mechanism of action is likely to cause a side effect in treating a specific manifestation of the patient's condition as evidenced by the classified anonymized patient health record data.

12. The method of claim 11, wherein the anonymized patient health record data includes (i) patient demographics, (ii) measurements of vital signs, (iii) physiological monitor data, (iv) the ward in which the patient is staying, (v) diagnosis and treatment information, (vi) lab test results, (vii) medication data, (viii) patient outcome information, (ix) clinical notes, and/or (x) patient medical history.

13. The method of claim 11, wherein the anonymized patient health record data reflects the nature of the patient population served by the hospital or clinic in terms of patient demographics, rates of disease incidence, and/or treatment practices.

14. The method of claim 14, wherein the anonymized patient health record data is sourced from a database of the plurality of patients, a database of one or more care centers and patient populations, or from a database of multiple care centers and patient populations.

15. The method of claim 11, wherein the anonymized patient health record data is collected at a standard interval.

16. The method of claim 11, wherein the anonymized patient health record data includes at least one gold standard patient data that identifies that patient is progressing through a disease pathway for which the drug is expected to be effective.

17. The method of claim 16, wherein the at least one gold standard patient data includes a specific progression through the disease pathway.

18. The method of claim 11, wherein the anonymized patient health record data continually improve as new data becomes available.

19. The method of claim 11, wherein the classified anonymized patient health record data includes an operating point that balances measurements of specificity and sensitivity in order to effectively treat as many patients as possible.

20. The method of claim 11, wherein the drug treatment is administration of drotrecogin alfa, tozadenant, vosaroxin, momelotinib or resatorvid.
